(19) United States
(12) Patent Application Publication
(10) Pub. No.: US 2014/0066696 A1
(43) Pub. Date: Mar. 6, 2014

(54) METHOD AND SYSTEM FOR DETERMINING THE PRESSURE OF A FLUID IN A SYRINGE, AN ACCESS PORT, A CATHETER, AND A GASTRIC BAND

(71) Applicant: Allergan, Inc., Irvine, CA (US)

(72) Inventors: Sean Snow, Carpinteria, CA (US); Mike Augarten, Goleta, CA (US); Christian Y. Perron, Ventura, CA (US); Ethan Franklin, Goleta, CA (US); Robert E. Hoyt, Jr., Santa Barbara, CA (US); Marcos Borrell, Goleta, CA (US); Babak Honaryar, Orinda, CA (US)

(73) Assignee: Allergan, Inc., Irvine, CA (US)

(21) Appl. No.: 14/075,789

(22) Filed: Nov. 8, 2013

Related U.S. Application Data
(63) Continuation of application No. 12/769,516, filed on Apr. 28, 2010, now abandoned.

(51) Int. Cl. A61F 5/00; A61B 19/00

(52) U.S. Cl. CPC A61F 5/0056; A61F 5/0059; A61B 19/5225
USPC 600/37

(57) ABSTRACT

A method and system for determining pressure in a syringe, and more specifically to a syringe pressure accessory which can be connected to a syringe to determine pressure in a syringe and a gastric band. The syringe pressure accessory can detect a pressure of a syringe and/or a gastric band and digitally display the pressure. The syringe pressure accessory can include a durable unit and a disposable unit. The disposable unit can be disposed of after a single use, while the durable unit can be reused with multiple disposable units. The syringe pressure accessory can also include a syringe attachment unit and one or more display units for a caretaker or a patient. The display unit can be wirelessly connected to the syringe attachment unit to display a pressure chart or the results of various analysis of the pressure data. Markers can be added to the pressure chart.

This application is a continuation of U.S. patent application Ser. No. 12/769,516, filed Apr. 29, 2010, the entire contents of which is incorporated herein by this reference.

### FIELD

The present invention generally relates to a method and system for determining pressure in a syringe, and more specifically to a syringe pressure accessory which can be connected to a syringe to determine the pressure of a fluid in a syringe, an access port, a catheter, and a gastric band.

### BACKGROUND

Adjustable gastric banding apparatus have provided an effective and substantially less invasive alternative to gastric bypass surgery and other conventional surgical weight loss procedures. Despite the positive outcomes of invasive weight loss procedures, such as gastric bypass surgery, it has been recognized that sustained weight loss can be achieved through a laparoscopically-placed gastric band, for example, the LAP-BAND® (Allergan, Inc., Irvine, Calif.) gastric band or the LAP-BAND APO (Allergan, Inc., Irvine, Calif.) gastric band. Generally, gastric bands are placed about the cardia, or upper portion, of a patient's stomach forming a stoma that restricts the passage of food into a lower portion of the stomach. When the stoma is of an appropriate size that is restricted by a gastric band, food held in the upper portion of the stomach provides a feeling of satiety or fullness that discourages overeating. Unlike gastric bypass procedures, the gastric band apparatus are reversible and require no permanent modification to the gastrointestinal tract.

Over time, a stoma created by a gastric band may need adjustment in order to maintain an appropriate size, which is neither too restrictive nor too passive. Accordingly, prior art gastric band systems provide a subcutaneous fluid access port connected to an expandable or inflatable portion of the gastric band. By adding fluid to or removing fluid from the inflatable portion by means of a hypodermic needle inserted into the access port, the effective size of the gastric band can be adjusted to provide a tighter or looser constriction.

However, adjustment of the gastric band is conventionally performed by a syringe. The amount of fluid added by the syringe is often determined by a visual inspection of the syringe by the user. Such methods are often inaccurate since the user may not be able to determine accurately an amount of fluid injected into the gastric band through only a visual inspection of the syringe. Furthermore, the user may not know the precise pressure of the gastric band and thus may add too much or too little fluid to the gastric band. Detection of such pressure can be costly and time consuming since conventional pressure detection apparatuses will need to be sterilized after each use or disposed of after each use. Complete disposal of the pressure detection apparatus can be expensive and cost-prohibitive.

Some pressure detection apparatuses used in conjunction with syringes have also been disclosed, but these devices suffer from certain disadvantages. For example, Nolan, U.S. Pat. No. 5,808,203, generally discloses a pressure measuring device for a syringe, but Nolan integrates the pressure sensor as part of the syringe.

Reilly, U.S. Pat. No. 4,370,982, generally discloses an apparatus for measuring and detecting the pressure in a syringe being injected into a closed system. However, Reilly integrates the pressure sensor as part of the syringe.

Dlugos et al., U.S. Pat. Pub. No. 2008/0015406 generally discloses an external pressure sensing system. Dlugos discloses that a pressure sensing portion comprises a reusable sensor portion and a disposable cap portion. The reusable sensor portion and the disposable cap portion are configured to selectively engage one another. When coupled with the reusable sensor portion, the disposable cap portion is in fluid communication with the reusable sensor portion, such that pressure of fluid within tubing may be communicated to the reusable sensor portion via the disposable cap portion. In one embodiment, the disposable cap portion comprises the pressure dome described in Adolfs et al., U.S. Pat. No. 6,725,726. The reusable sensor portion comprises a pressure port, which is configured to receive such fluid pressure communications from the disposable cap portion. For instance, the pressure port may comprise a diaphragm or other structure suited for receiving fluid pressure communications. The reusable sensor portion further comprises a pressure sensor, such as a transducer, which is configured to provide pressure data via a cable to an interface component. The interface component is operable to process such pressure data and communicate it to a display device via a cable. However, Dlugos suffers from the drawback that it does not disclose the use of a disposable membrane, that the diaphragm is a reusable membrane, or that the diaphragm in the reusable sensor portion should contact a diaphragm in the disposable cap portion. This can, for example, result in inaccurate pressure information and/or contamination of the fluid being injected into the gastric band.

Thus, there is a need for a method and system for determining pressure in a syringe, and more specifically to a syringe pressure accessory which can be connected to a syringe to determine pressure in a syringe, an access port, a catheter, and a gastric band.

### SUMMARY

The present invention is directed to a method and system for determining pressure in a syringe, and more specifically to a syringe pressure accessory which can be connected to a syringe to determine pressure in a syringe, an access port, a catheter, and a gastric band.

In one embodiment, the present invention is a syringe pressure accessory including a pressure sensor configured to be connected to a syringe and to detect pressure data corresponding to pressure in the syringe, and a processor connected to the pressure sensor and to analyze the pressure data to determine the pressure in the syringe.

In one embodiment, the present invention is a syringe pressure accessory including a pressure sensor configured to be connected to a syringe and to detect pressure data corresponding to pressure of the fluid in the syringe, and a processor connected to the pressure sensor and to analyze the pressure data to determine the pressure of the fluid in the syringe.

In another embodiment, the present invention is a syringe pressure accessory including a pressure sensor configured to be connected to a syringe and to detect pressure data corresponding to pressure in the syringe, a processor connected to the pressure sensor and to analyze the pressure data to determine the pressure in the syringe, and an electrical parameter measuring system connected to the processor, the electrical parameter measuring system detecting electrical parameter data indicating a location of the syringe relative to a gastric band, or a port for a gastric band.

In another embodiment, the present invention is a syringe pressure accessory including a disposable unit configured to be connected to a syringe and including a pressure sensor detecting pressure data, and a durable unit configured to be connected to the disposable unit to determine the pressure in the syringe.

In yet another embodiment, the present invention is a syringe pressure accessory including a syringe attachment unit configured to be connected to a syringe and to detect pressure data of the syringe, and a first digital display unit configured to wirelessly connect to the syringe attachment unit, the first digital display unit configured to indicate a pressure corresponding to the pressure data of the syringe.

In still yet another embodiment, the present invention is a method for determining pressure in multiple syringes including using a first disposable unit to detect pressure data of a first syringe, and using a durable unit to analyze the pressure data of the first syringe to determine the pressure in the first syringe.

### BRIEF DESCRIPTION OF THE DRAWINGS

FIG. 1 illustrates syringe pressure accessory according to an embodiment of the present invention.

FIG. 2 illustrates a box diagram of a syringe pressure accessory according to an embodiment of the present invention.

FIG. 3 illustrates a durable unit and a disposable unit for a syringe pressure accessory according to an embodiment of the present invention.

FIG. 4 illustrates a durable unit and a disposable unit according to an embodiment of the present invention.

FIG. 5 illustrates a disposable unit according to an embodiment of the present invention.

FIG. 6 illustrates a cross-section of a disposable unit according to an embodiment of the present invention.

FIG. 7 illustrates a durable unit and a disposable unit according to an embodiment of the present invention.

FIG. 8 illustrates a durable unit and a disposable unit according to an embodiment of the present invention.

FIG. 9 illustrates a durable unit and a disposable unit according to an embodiment of the present invention.

FIG. 10 illustrates a cross-section of a durable unit and a disposable unit according to an embodiment of the present invention.

FIG. 11 illustrates a syringe pressure attachment unit and a display unit according to an embodiment of the present invention.

FIG. 12 illustrates a box diagram of a display/receiver unit according to an embodiment of the present invention.

FIG. 13 illustrates a box diagram of a syringe pressure accessory unit according to an embodiment of the present invention.

FIG. 14 illustrates a process according to an embodiment of the present invention.

FIG. 15 illustrates a process according to an embodiment of the present invention.

FIG. 16 illustrates a process according to an embodiment of the present invention.

FIG. 17 illustrates a process according to an embodiment of the present invention.

FIG. 18 illustrates a process according to an embodiment of the present invention.

### DETAILED DESCRIPTION

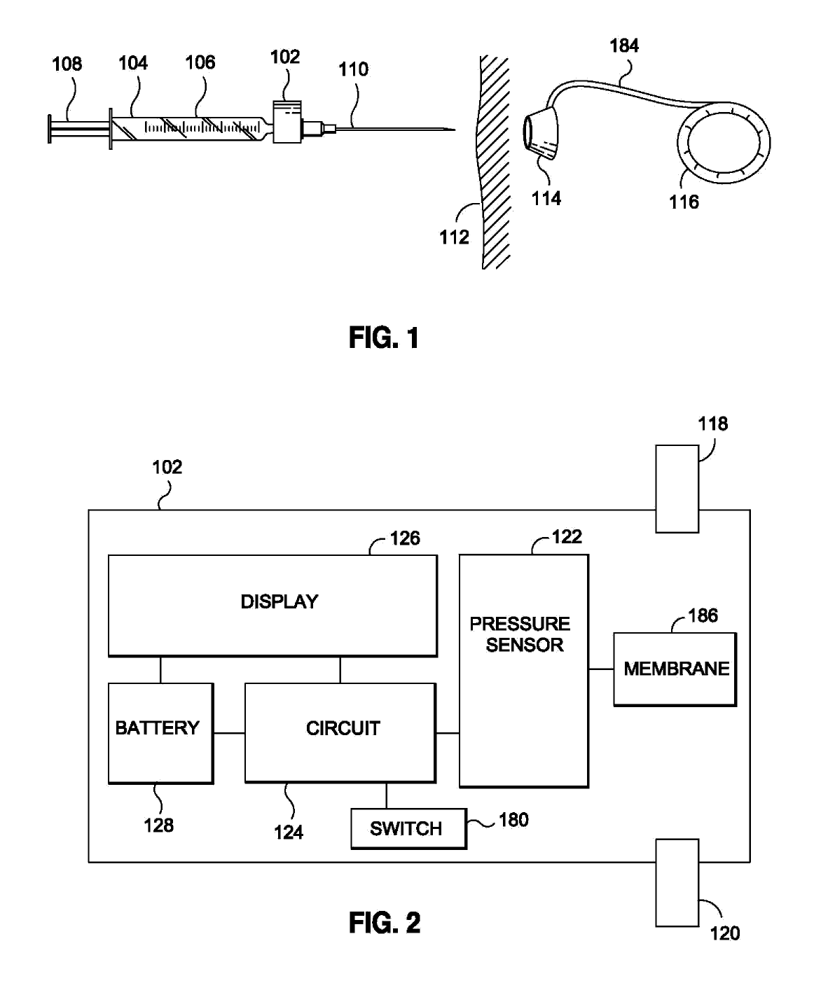

As seen in FIG. 1, in one embodiment, the present invention can include a syringe pressure accessory 102 attached to a syringe 104. The syringe 104 can include, for example, a barrel 106, a pump 108, and/or an optional needle 110. The syringe pressure accessory 102 can be attached, for example, to the barrel 106 and/or the needle 110. Although the needle 110 is shown in FIG. 1, a tube, a catheter, or other connection device can be used instead of or in conjunction with the needle 110.

In FIG. 1, a gastric band 116 is connected to a port 114 by a tube 184. The port 114 can be, for example, an access port. The gastric band 116, the port 114, and/or the tube 184 are implanted within a patient and can lie beneath a skin layer 112. The syringe 104 can connect to the port 114 by piercing the skin layer 112 with the needle 110. The barrel 108 can be depressed to inject fluid into the port 114 and to the gastric band 116 or remove fluid from the port 114 and the gastric band 116.

One embodiment of the syringe pressure accessory 102 can be seen, for example, in FIG. 2. The syringe pressure accessory 102 can include, for example, a connection unit 118, a connection unit 120, a pressure sensor 122, a circuit 124, a display 126, a battery 128, a membrane 186, and/or a switch 180.

The syringe pressure accessory can connect to the syringe 104 using the connection unit 118 and/or the connection unit 120. In one embodiment, the connection unit 118 can be a male connection or a female connection unit, and the connection unit 118 can be a male connection unit or a female connection unit. Furthermore, the barrel 106 can be connected to the syringe pressure accessory at the connection unit 118 or the connection unit 120. In addition, the needle 110 can be connected to the connection unit 118 or the connection unit 120.

The membrane 186 can be connected to the pressure sensor 122. In one embodiment, the membrane 186 is in fluid connection with the syringe 104 and can be used, for example, to determine the pressure of the syringe 104. The membrane 186 can also be used, for example, to determine the pressure in the port 114, the gastric band 116, the needle 110, and/or a catheter connecting the syringe 104 to the port 114. The pressure in the syringe 104 can also correspond, for example, to the pressure in the port 114 and/or the gastric band 116.

The pressure sensor 122 can be connected, for example, to the membrane 186. The pressure sensor 122 can use the membrane 186 to detect pressure data. The pressure data can correspond, for example, to the pressure in the syringe 104. The pressure in the syringe 104 can correspond, for example, to the pressure in the port 114 and/or the gastric band 116.

The switch 180 is connected to the circuit 124 and can supply an on signal or an off signal to the circuit 124. The display 126 is connected to the circuit 124 and can display various pieces of information that are relevant to a user using the syringe pressure accessory. The battery 128 is connected, for example, to the display 126 and/or the circuit 124.

The battery 128 can store power and also supply power to the display 126, and/or the circuit 124. The battery 128 can also supply power to the pressure sensor 122 and/or any other electrical device in the syringe pressure accessory 102. In addition, the battery 128 can be, for example, rechargeable.

The circuit 124 can be connected, for example, to the switch 180, the pressure sensor 122, and/or the display 126. The circuit 124 can turn on or off the syringe pressure accessory based on whether the circuit 124 receives an on signal or an off signal from the switch 180. The circuit 124 can also receive the pressure data from the pressure sensor 122, and analyze the pressure data to determine the pressure in the syringe 104 and/or the gastric band 116. In one embodiment, the circuit 124 can also control the display 126 to display the pressure of the syringe 104 and/or the gastric band 116. The circuit 124 can include, for example, a micro controller. The micro controller can be, for example, a processor.

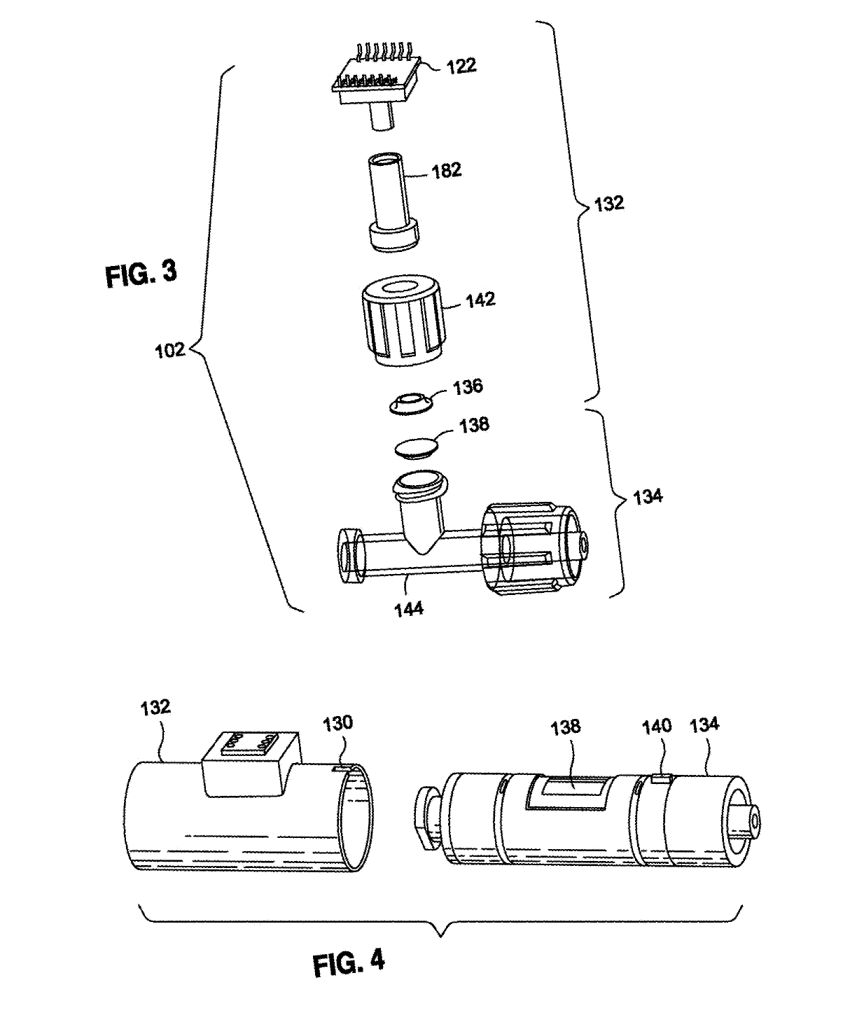

In one embodiment, the syringe pressure accessory 102 can include, for example, a durable unit 132 and a disposable unit 134, as seen in FIG. 3. The durable unit 132 can be connected, for example, to the disposable unit 134. The durable unit 132 can be reused while the disposable unit 134 can be disposed after each use. The disposable unit 134 can be disposed, for example, along with a syringe.

The durable unit 132 can include, for example, a pressure sensor 122, a flanged sleeve 182, a luer spin lock 142, and/or a reusable membrane 136. The luer spin lock 142 can be configured to secure the durable unit 132 to the disposable unit 134. The reusable membrane 136 can contact the disposable unit 134 and allow the pressure sensor 122 to detect pressure data from the disposable unit 134. The flanged sleeve 182 provides the pressure sensor 122 access to the reusable membrane 136. Although not shown in FIG. 3, the durable unit 132 can also include a processor to analyze the pressure data to determine the pressure in the syringe and/or the gastric band 116. Thus, the pressure sensor 122 can be, for example, a reusable pressure sensor.

The disposable unit 134 can include, for example, a t-connector 144, and/or a disposable membrane 138. The t-connector 144 can connect the disposable unit to a syringe and/or the durable unit 132. The t-connector 144 can also allow the disposable membrane 138 to be in fluid communication with the syringe and/or a gastric band.

In operation, the disposable membrane 138 can be in fluid communication with the syringe, an access port, a catheter, and/or a gastric band. The reusable membrane 136 can contact the disposable membrane 138 such that pressure in the syringe, the access port, the catheter, and/or the gastric band can be indicated and/or detected by the reusable membrane 136. The pressure sensor 122 can generate pressure data based on the pressure detected by the reusable membrane 136. Once the collection of the pressure data by the pressure sensor 122 is completed, the disposable unit 134 can be disposed along with the syringe. The process can then be repeated using another disposable unit 134 and another syringe, but with the same durable unit 132 for a new patient. Since the syringe is in fluid communication with the gastric band, the pressure of the syringe can be approximately equal to the pressure in the gastric band. Likewise, the pressure of the catheter and/or the access port can be approximately equal to the pressure in the gastric band. Thus, the pressure of the syringe, the catheter, and/or the access port can correspond, for example, to the pressure of the gastric band.

FIG. 4 depicts another embodiment of the durable unit 132 and the disposable unit 134. As can be seen in FIG. 4, the disposable membrane 138 is exposed such that it can be in contact with the durable unit 132 to allow detection of the pressure within a syringe. The durable unit 132 includes a connection unit 130 which can be mated with a connection unit 140 to secure the durable unit 132 to the disposable unit 134. The connection unit 130 can be, for example, a notch, while the connection unit 140 can be, for example, a protrusion.

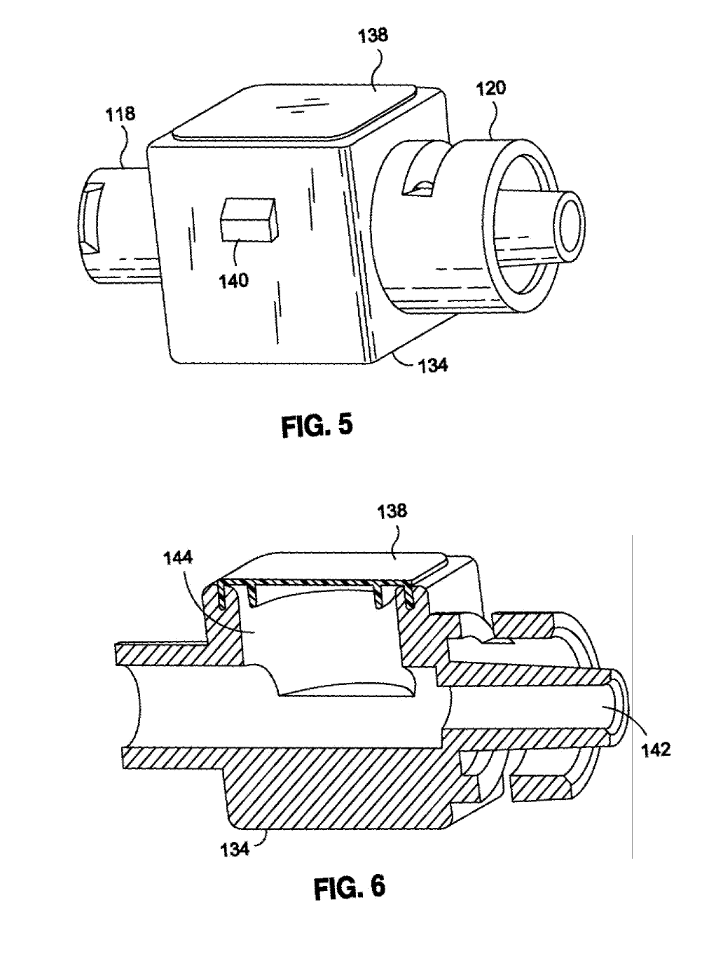

FIG. 5 depicts another embodiment of the disposable unit 134. As can be seen, the disposable membrane 138 is exposed such that it can be in contact with the durable unit 132 (not shown) to allow detection of the pressure within a syringe. The connection unit 140 can be mated, for example, to a retention clip (not shown) in the durable unit 132. The connection unit 118 and the connection unit 120 can be used, for example, to connect the disposable unit 134 to a syringe.

FIG. 6 depicts a cross-section of the disposable unit 134 shown in FIG. 5. As can be seen, the disposable unit 134 includes a channel 142 and a chamber 144. The channel 142 can be in fluid communication with the syringe when the disposable unit 134 is connected to the syringe. The syringe can be in fluid communication with a gastric band. The channel 142 and the disposable membrane 138 are also in fluid connection with the chamber 144. Thus, when the disposable unit 134 is connected to the syringe, and the syringe is in fluid communication with the gastric band, the disposable membrane 138 can indicate a pressure of the syringe and/or the gastric band. The disposable unit 134 can also be in fluid communication with an access port and/or a catheter to indicate a pressure of the syringe and/or the gastric band.

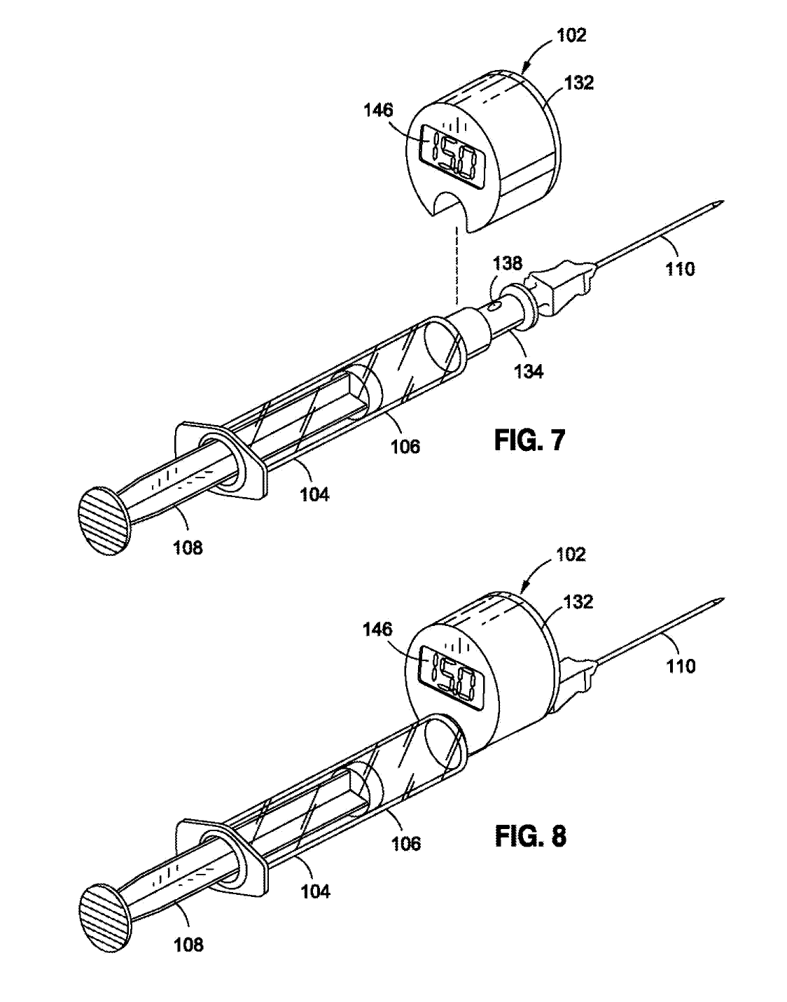

FIG. 7 depicts another embodiment of the syringe pressure accessory 102 including the durable unit 132 and the disposable unit 134. As can be seen, the durable unit 132 includes, for example, a display 146. The display 146 can indicate, for example, the pressure of the syringe 104 and/or the gastric band 116. In one embodiment, the display 146 is a digital display. As can be seen, the disposable unit 134 is connected to the syringe 104. Furthermore, as seen in FIG. 7 and FIG. 8, the durable unit 132 can be connected to the disposable unit 134.

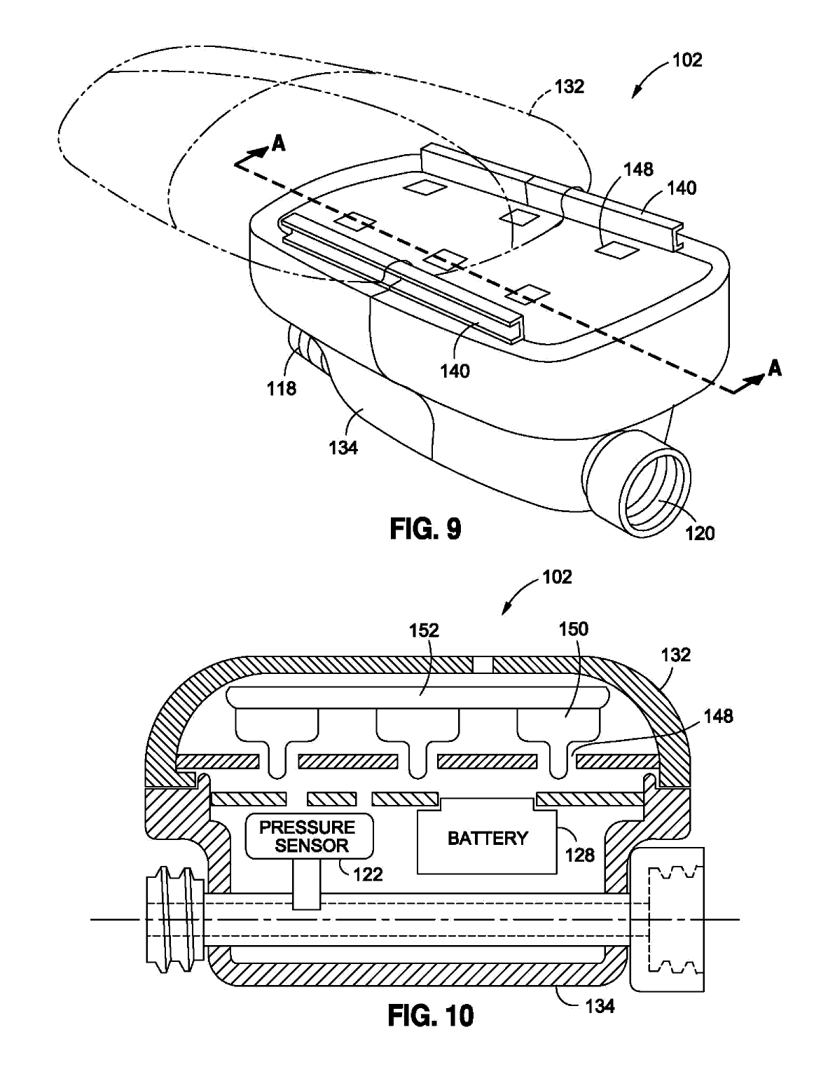

FIG. 9 and FIG. 10 depict another embodiment of the syringe pressure accessory unit 102 including a durable unit 132 and a disposable unit 134. In FIG. 9 and FIG. 10, the pressure sensor 122 now resides in the disposable unit 134 instead of the durable unit 132. The pressure sensor 122 can be powered, for example, by the battery 128. The battery 128 can contain sufficient power, for example, to power the pressure sensor 122 before the pressure sensor 122 and the battery 128 are disposed of. The location of the pressure sensor 122 can obviate the need for a membrane, and thus the durable unit 132 and the disposable unit 134 can be constructed without a membrane. Thus, the syringe pressure accessory 102 can utilize a pressure sensor 122 in the disposable unit 134 that is disposable. The pressure sensor 122 can directly determine pressure data from the syringe.

A processor 152, however, may still reside in the durable unit 132. Furthermore, the disposable unit 134 can include metal contacts 148 and the durable unit 132 can include metal contacts 150. The metal contacts 148 and the metal contacts 150 are configured to mate with each other allowing the transfer of pressure data from the pressure sensor 122 in the disposable unit 134 to the processor 152 in the durable unit 132. In addition, as seen in FIG. 9, the connection unit 140 can be utilized to secure the durable unit 132 to the disposable unit 134.

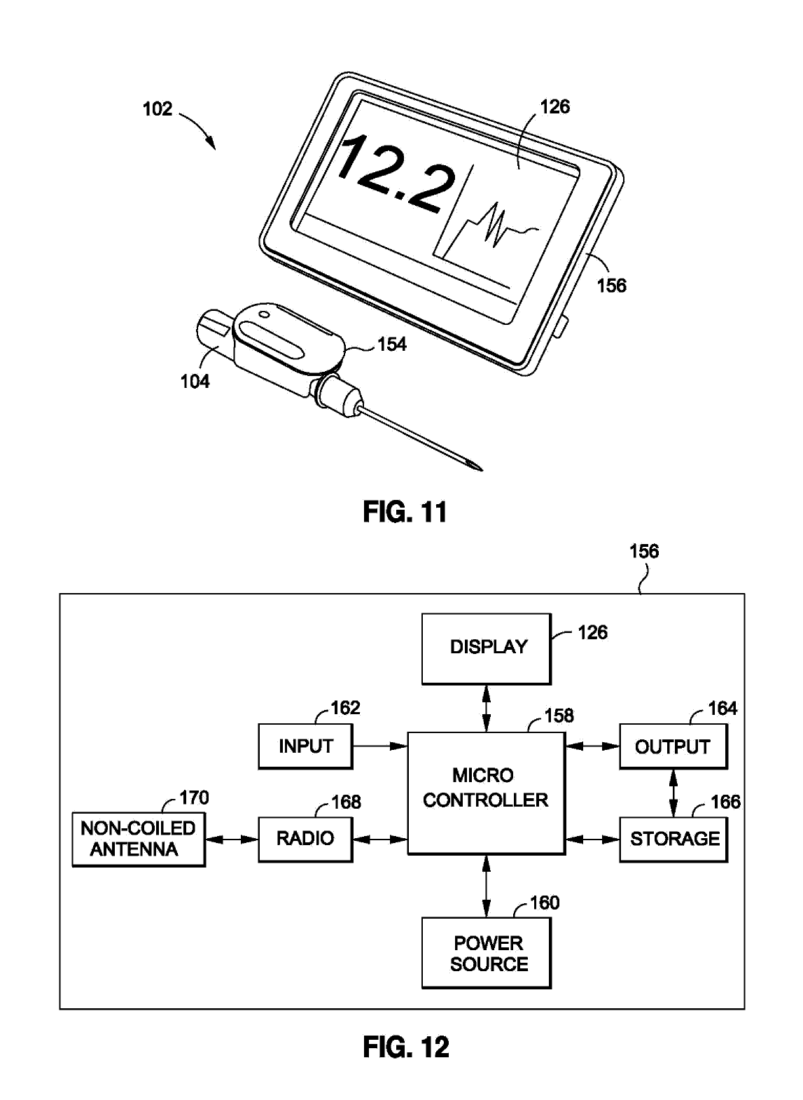

FIG. 11 depicts another embodiment of the syringe pressure accessory 102. In FIG. 11, the syringe pressure accessory 102 can include, for example, a syringe attachment unit 154 and a display unit 156. The display unit 156 can also be, for example, a receiver unit. The syringe attachment unit 154 can be attached to the syringe 104 and determine pressure data corresponding to the pressure within the syringe 104 and/or a gastric band. The syringe attachment unit 154 can wirelessly transmit the pressure data to the display unit 156. The display unit 156 can include, for example, a display 126. The display unit 156 can receive the pressure data and display information regarding the pressure data, such as a numerical representation of the pressure data or a pressure chart on the display 126.

FIG. 12 depicts an embodiment of the display unit 156. As seen in FIG. 12, the display unit 156 includes a display 126, a micro controller 158, a power source 160, an input 162, an output 164, a storage device 166, a radio 168, and/or a non-coiled antenna 170.

The display 126 is connected to the micro controller 158 and can display, for example, information related to the pressure of the syringe and/or the gastric band. The input 162 is connected, for example, to the micro controller 158. The input 162 can be, for example, a button, a mouse, a keyboard, a tilt sensor, a light sensor, a touch sensor, a security feature, and/or any other type of device which can supply information or data to the micro controller 158.

The output 164 can be connected, for example, to the micro controller 158 and/or the storage device 166. The output 164 can allow for example, the output of data such as pressure data and/or pressure charts. In one embodiment, the output 164 can transmit the pressure data and/or the pressure charts to the storage 166 for storage.

The storage device 166 is connected to the micro controller 158 and/or the output 164. The storage device 166 can store, for example, the pressure data and/or the pressure charts. The storage can be, for example, a flash memory and/or a hard drive.

The power source 160 is connected to the micro controller 158. The power source 160 can be, for example, a battery, and/or a medical or a non-medical grade power supply. The power source 160 can supply power to the micro controller 158 and/or any other electrical device in the syringe pressure accessory.

The radio 168 is connected, for example, to the micro controller 158 and/or the non-coiled antenna 170. The radio allows for wireless transmission and/or reception of data, such as the pressure data.

The non-coiled antenna 170 is connected, for example, to the radio 168. The non-coiled antenna 170 aids in the wireless transmission and/or reception of data, such as the pressure data. The non-coiled antenna can be, for example, easier and more cost-effective to implement than a coiled antenna.

The micro controller 158 is connected, for example, to the display 126, the input 162, the output 164, the storage 166, the power source 160, and/or the radio 168. The micro controller 158 can receive, for example, the pressure data from the input 162 and/or the radio 168. The micro controller 158 can analyze the pressure data to generate the pressure charts. The micro controller 158 can also output the pressure data and/or the pressure charts to the output 164 and/or the storage 166. In addition, the micro controller 158 can instruct the display 126 to display the pressure data and/or the pressure charts.

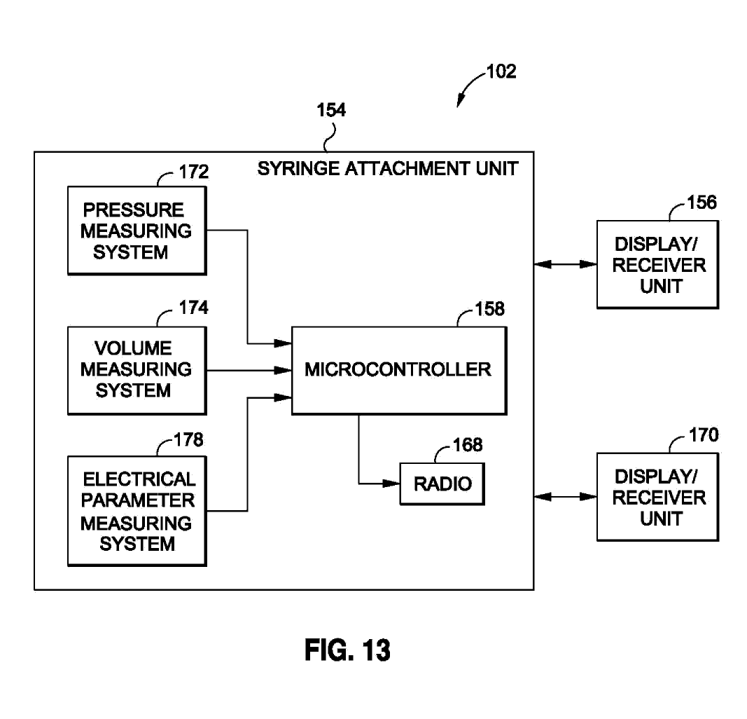

Another embodiment of the syringe pressure accessory 102 can be seen, for example, in FIG. 13. As seen in FIG. 13, the syringe pressure accessory 102 includes a syringe attachment unit 154, a display unit 156, and a display unit 170. The display unit 156 and the display unit 170 can also be, for example, receiver units. As seen in FIG. 13, multiple display units are used. The syringe attachment unit 154 can be connected, for example, to a syringe. The syringe can be in fluid communication with an access port, a catheter, and/or a gastric band.

The syringe attachment unit 154 can be configured to exchange data with the display unit 156 and/or the display unit 170. The syringe attachment unit 154 can include, for example, a radio 168, a pressure measuring system 172, a volume measuring system 174, and/or an electrical parameter measuring system 178.

The radio 168 can be connected, for example, to the micro controller 158. The radio 168 can facilitate, for example, wireless communications between the micro controller 158 and the display unit 156 and/or the display unit 170.

The pressure measuring system 172 is connected, for example, to the micro controller 158. The pressure measuring system 172 can detect, for example, pressure data corresponding to a pressure of the syringe. The pressure of the syringe can correspond, for example, to the pressure of the gastric band.

The volume measuring system 174 is connected, for example, to the micro controller 158. The volume measuring system 174 can detect, for example, volume data indicating a volume of fluid dispensed by the syringe. The volume of fluid dispensed by the syringe can correlate, for example, with the volume of fluid received by the gastric band from the syringe. The volume measuring system 174 can be, for example, a differential pressure sensor, a Coriolis effect sensor, a paddle wheel, an optical sensor, and/or any other type of sensor that can detect volume of a fluid.

The electrical parameter measuring system 178 is connected, for example, to the micro controller 158. The electrical parameter measuring system 178 can detect, for example, electrical parameter data indicating a location of the syringe relative to a gastric band, or a port for a gastric band. The electrical parameter measuring system 178 can be, for example, an electrical sensor. The electrical parameter measuring system 178 can measure conductivity, resistance, breakdown voltage, and/or other electromagnetic properties over a range of frequencies, waveform shapes, voltage levels, and current levels. Such information can be used, for example, to determine a location of the needle of the syringe relative to the body tissue, determine the position of the needle of the syringe relative to the port, determine the status of the port, determine the health of the tissue, determine the tissue layer composition and depth, determine the status of the fluid in the port. The status of the port can include, for example, level of tissue ingrowths or encapsulation. The status of the fluid in the port can include, for example, an amount of bacterial growth and/or the pH level of fluids in the port.

The micro controller 158 can transmit, for example, the pressure data, the volume data, and/or the electrical parameter data to the display unit 156 and/or the display unit 170. The micro controller 158 can also analyze the pressure data, the volume data, and/or the electrical parameter data to generate various charts or images which can also be transmitted to the display unit 156, and/or the display unit 170. The display unit 156 and/or the display unit 170 can display some or all of the pressure data, the volume data, the electrical parameter data, and/or the various charts or images.

In one embodiment, the display unit 156 is configured to be used, for example, by a doctor or caretaker, while the display unit 170 is configured to be used, for example, by a patient. The display unit 156 can be more sophisticated and allow the display of a greater amount of information. The display unit 170 can be less sophisticated and allow the display of a reduced amount of information. Furthermore, in one embodiment, the display unit 156 can control the display of information on the display unit 170.

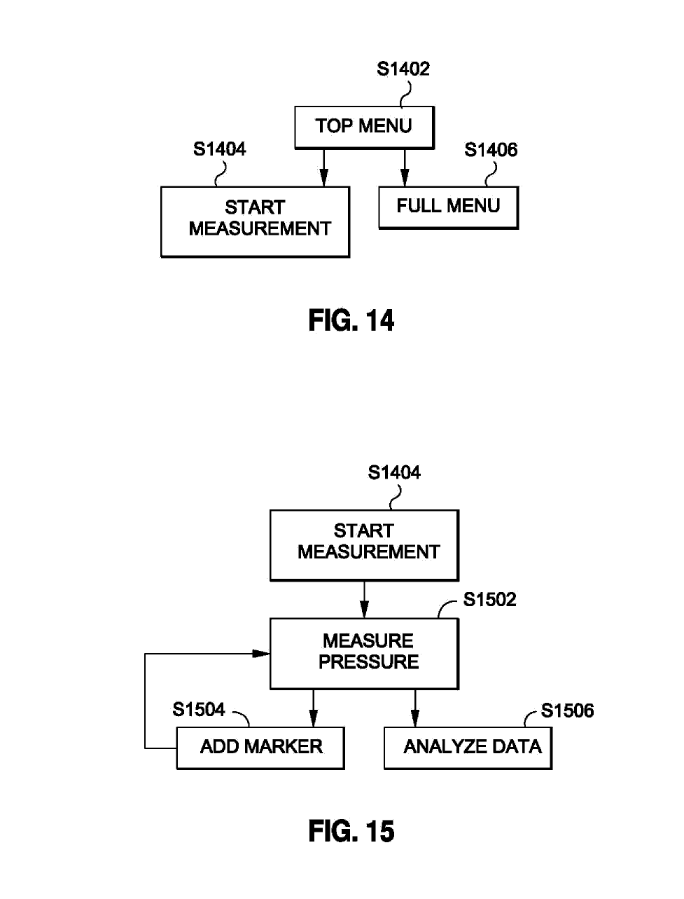

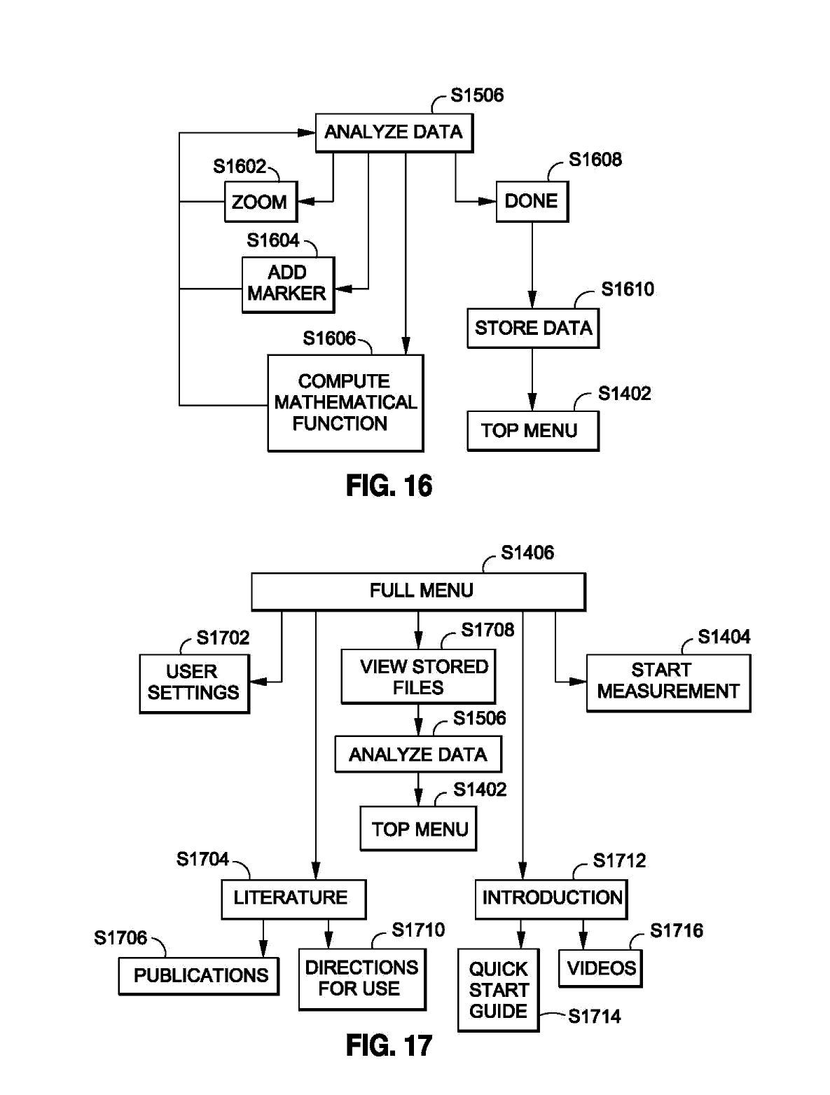

In one embodiment, a syringe pressure accessory including, for example, the display unit 156 and/or the syringe attachment unit 154 can perform a process such as those depicted in FIG. 14, FIG. 15, FIG. 16, and/or FIG. 17. In Step S1402, a top menu can be displayed. For example, the micro controller 158 can instruct the display 126 to display a top menu including a start measurement button and a full menu button. If the start measurement button is pressed, the process proceeds to Step S1404. Otherwise, the process proceeds to Step S1406. In Step S1404, the start measurement process begins as shown in FIG. 15.

In Step S1502, pressure is measured. For example, the pressure measuring system 172 can measure medical data such as pressure data, volume data, and/or electrical parameter data, which are transmitted to the display unit 156. The display unit 156 can display the medical data and/or a medical chart from the medical data. Furthermore, the display unit 156 can display, for example, a marker button and an analyze data button. If the marker button is selected, the process proceeds to Step S1504. In Step S1504, a marker is added. For example, the display 126 can display a marker on the medical chart. The marker can indicate, for example an event. The marker can be, for example, a line, a rectangle, a triangle, a circle, or any other icon that can indicate an event. The process then repeats at Step S1502.

If the analyze data button is selected, the process proceeds to Step S1506. In Step S1506, the analyze data process begins as shown in FIG. 16. For example, the display 126 can display a zoom button, an add market button, a computer mathematical function button, and/or a done button. If the zoom button is selected, the process proceeds to Step S1602. In Step S1602, a zoom function is implemented. For example, the medical chart can be zoomed in. The process then repeats at Step S1506.

If the add marker button is selected, the process proceeds to Step S1604. In step S1604, a marker is added. The marker can indicate, for example an event. The marker can be, for example, a line, a rectangle, a triangle, a circle, or any other icon that can indicate an event. The process then repeats at Step S1506.

If the compute mathematical function button is selected, the process proceeds to Step S1606. In Step S1606, mathematical functions are computed. For example, data analysis can be performed on the medical data and the results of the data analysis can be displayed. Such analysis can include, for example, trends, valleys, peaks, recommendations, or any other type of analysis which may be beneficial to a doctor, caretaker, or patient.

If the done button is selected, the process proceeds to Step S1608. In Step S1608, the process prepares for the completion of the analyze data process. In Step S1610, data is stored. For example, the micro controller 158 can store the medical data. Alternatively the medical data can be transmitted to an external computer for storage. The process then repeats at Step S1402 (FIG. 14).

If the full menu button is selected in FIG. 14, the process proceeds to the full menu process in Step S1406, as shown in FIG. 16. In Step S1406 a full menu is displayed. The full menu can include, for example, a user settings button, a literature button, a view stored files button, an introduction button, and/or a start measurement button. If the user settings button is selected, the process proceeds to Step S1702. In Step S1702 user settings are displayed. For example, the user settings can allow the user to set touch sensitivity of the display 126, a brightness or contrast of the display 126, a layout of the display 126, or any other customizable feature for the display unit 156 and/or the syringe attachment unit 154. The process then repeats at Step S1406.

If the literature button is selected, the process proceeds to Step S1704. In Step S1704, literature options are displayed. For example, literature options such as a publications button and/or directions for use button can be displayed on the display 126. If the publications button is selected, the process proceeds to Step S1706.

In Step S1706, publications are displayed. For example, publications which may be helpful to the user may be displayed in the display 126. In addition, the user may select which publication to display. If the directions for use button is selected, the process proceeds to Step S1710. In Step S1710, the directions for use are displayed. For example, the display 126 can display the directions for using the syringe pressure accessory 102, the display unit 156, and/or the syringe attachment unit 154.

If the view stored files button is selected, the process proceeds to Step S1708. In Step S1708, the stored files are displayed. For example, medical data which are stored can be displayed in the display 126. The process then proceeds to Step S1506. In Step S1506, the data can be analyzed, as previously discussed above, and shown in FIG. 16. The process then proceeds to Step S1402. In Step S1402, the top menu is displayed as previously discussed above, and shown in FIG. 14.

If the introduction button is selected, the process proceeds to Step S1712. In Step S1712, an introduction is displayed. For example, a quick start guide button and/or a videos button can be displayed. If the quick start guide button is selected, the process proceeds to Step S1714.

In Step S1714, a quick start guide is displayed. For example, the display 126 can display a quick start guide for using the syringe pressure accessory 102, the display unit 156, and/or the syringe attachment unit 154. If the videos button is selected, the process proceeds to Step S1716. In Step S1716, videos are displayed. For example, videos can be displayed on the display 126 for using the syringe pressure accessory 102, the display unit 156, and/or the syringe attachment unit 154. However, other videos many also be displayed.

If the Start measurement button is selected, the process proceeds to Step S1404 as described above, and shown, for example, in FIG. 15.

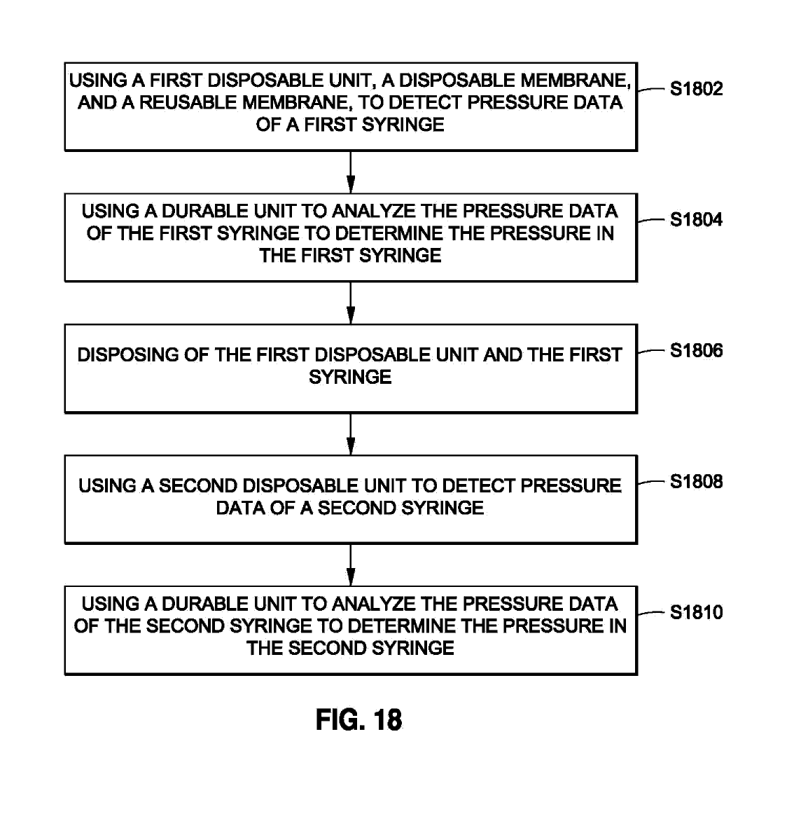

In one embodiment, the present invention is a process as shown in FIG. 18. In Step S1802, a first disposable unit, a disposable membrane, and a reusable membrane are used to detect pressure data of a first syringe. For example, as shown in FIG. 3, the disposable unit 134, the disposable membrane 138, and a reusable membrane 136 can be used to detect pressure data of a syringe. In one embodiment, instead of using the disposable membrane 138 or the reusable membrane 136, only a disposable pressure sensor 122 is used.

In Step S1804, a durable unit can be used to analyze the pressure data of the first syringe to determine the pressure in the first syringe. For example, the durable unit 132 can be used to analyze the pressure data of the syringe to determine the pressure in the syringe.

In Step S1806, the first disposable unit and the first syringe can be disposed. For example, the disposable unit 134 and the syringe can be disposed. In Step S1808, a second disposable unit is used to detect pressure data of a second syringe. For example, another disposable unit 134 is used to detect the pressure data of a new syringe, aside from the disposable unit 134 and the syringe that were disposed. In Step S1810, a durable unit is used to analyze the pressure data of the second syringe to determine the pressure in the second syringe. For example, the same durable unit 132 is used to analyze the pressure data of the new syringe to determine the pressure in the new syringe.

The terms “a,” “an,” “the,” and similar referents used in the context of describing the present invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the present invention and does not pose a limitation on the scope of the present invention otherwise claimed. No language in the specification should be construed as indicating any non-claimed element essential to the practice of the present invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, certain references have been made to patents and printed publications throughout this specification. Each of the above-cited references and printed publications are individually incorporated herein by reference in their entirety.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

In closing, it is to be understood that the embodiments of the present invention disclosed herein are illustrative of the principles of the present invention. Other modifications that may be employed are within the scope of the present invention. Thus, by way of example, but not of limitation, alternative configurations of the present invention may be utilized in accordance with the teachings herein. Accordingly, the present invention is not limited to that precisely as shown and described.

What is claimed is:

1. A syringe pressure accessory for use in conjunction with a gastric band for the treatment of obesity, the syringe pressure accessory comprising:
a disposable membrane in fluid communication with a fluid in the syringe;
a reusable membrane in contact with the disposable membrane for indicating a pressure of the fluid in the syringe;
a reusable pressure sensor connected to the reusable membrane for detecting the indicated pressure of the fluid in the syringe; and
a processor attached to the reusable pressure sensor for analyzing the pressure of the fluid in the syringe.

2. The syringe pressure accessory of claim 1 further comprising a battery connected to the processor.

3. The syringe pressure accessory of claim 1 further comprising a switch connected to the processor to activate or deactivate the reusable pressure sensor.

4. The syringe pressure accessory of claim 1 further comprising a first connection unit configured to be connected to a first portion of the syringe.

5. The syringe pressure accessory of claim 4 further comprising a second connection unit configured to be connected to a second portion of the syringe.

6. The syringe of claim 5 wherein the first portion of the syringe includes a barrel of the syringe.

7. The syringe of claim 6 wherein the second portion of the syringe includes a needle of the syringe.

8. The syringe pressure accessory of claim 1 wherein the pressure in the syringe corresponds to pressure in a gastric band.

9. The syringe pressure accessory of claim 1 further comprising a volume measuring system connected to the processor, the volume measuring system detecting volume data indicating a volume of fluid added to a gastric band from the syringe.

10. The syringe pressure accessory of claim 1 further comprising an electrical parameter measuring system connected to the processor, the electrical parameter measuring system detecting electrical parameter data indicating a location of the syringe relative to a gastric band, a location of the syringe relative to a port for a gastric band, a status of the port for the gastric band, a health of a tissue, a tissue layer composition and depth, or a status of fluids in the port for the gastric band.

11. The syringe pressure accessory of claim 1 further comprising a digital display unit connected to the processor, the digital display unit indicating the pressure in the syringe.

12. The syringe pressure accessory of claim 1 wherein the digital display is configured to indicate the pressure in real-time.

13. The syringe pressure accessory of claim 1 wherein the digital display is configured to display a pressure chart based on the pressure data.

14. The syringe pressure accessory of claim 13 wherein the digital display is configured to add a marker to the pressure chart.

15. The syringe pressure accessory of claim 1 wherein the processor is configured to analyze the pressure data and the digital display is configured to display a result of the analysis of the pressure data.

16. A syringe pressure accessory comprising:
a pressure sensor configured to be connected to a syringe and to detect pressure data corresponding to pressure in the syringe;
a processor connected to the pressure sensor and to analyze the pressure data to determine the pressure in the syringe; and
an electrical parameter measuring system connected to the processor, the electrical parameter measuring system detecting electrical parameter data indicating a location of the syringe relative to a gastric band, a location of the syringe relative to a port for a gastric band, a status of the port for the gastric band, a health of a tissue, a tissue layer composition and depth, or a status of fluids in the port for the gastric band.

17. The syringe pressure accessory of claim 16 further comprising a battery connected to the circuit, and a switch connected to the processor to activate or deactivate the pressure sensor.

18. The syringe pressure accessory of claim 16 further comprising a first connection unit configured to be connected to a first portion of the syringe.

19. The syringe pressure accessory of claim 18 further comprising a second connection unit configured to be connected to a second portion of the syringe.

20. The syringe of claim 19 wherein the first portion of the syringe includes a barrel of the syringe, and wherein the second portion of the syringe includes a needle of the syringe.

21. A syringe pressure accessory comprising:
a disposable unit configured to be connected to a syringe and including a disposable membrane; and
a durable unit configured to be connected to the disposable unit to determine the pressure in the syringe.

22. The syringe pressure accessory of claim 21 wherein the durable unit includes a reusable membrane in contact with the disposable membrane.

23. The syringe pressure accessory of claim 22 wherein the durable unit includes a pressure sensor detecting pressure data using the reusable membrane.

24. The syringe pressure accessory of claim 23 wherein the pressure data corresponds to pressure in the syringe.

25. The syringe pressure accessory of claim 24 wherein the pressure in the syringe corresponds to pressure in a gastric band.

26. The syringe pressure accessory of claim 22 further comprising a processor connected to the pressure sensor, the processor analyzing the pressure data to determine the pressure in the syringe.

27. The syringe pressure accessory of claim 26 wherein the durable unit further includes a digital display unit, the digital display unit indicating the pressure in the syringe.

28. The syringe pressure accessory of claim 21 wherein the durable unit includes a non-coiled antenna.

29. The syringe pressure accessory of claim 21 wherein the disposable unit includes a pressure sensor detecting pressure data using the disposable membrane.

30. The syringe pressure accessory of claim 29 wherein the pressure data corresponds to pressure in the syringe.

31. The syringe pressure accessory of claim 30 wherein the pressure in the syringe corresponds to pressure in a gastric band.

32. The syringe pressure accessory of claim 29 wherein the durable unit includes a processor electrically connected to the pressure sensor, the processor analyzing the pressure data to determine the pressure in the syringe.

33. A syringe pressure accessory comprising:
a disposable unit configured to be connected to a syringe and including a pressure sensor for detecting pressure data; and
a durable unit configured to be connected to the disposable unit to determine the pressure in the syringe.

34. The syringe pressure accessory of claim 33 wherein the pressure data corresponds to pressure in the syringe.

35. The syringe pressure accessory of claim 34 wherein the pressure in the syringe corresponds to pressure in a gastric band.

36. The syringe pressure accessory of claim 33 wherein the durable unit includes a processor electrically connected to the pressure sensor, the processor analyzing the pressure data to determine the pressure in the syringe.

37. A syringe pressure accessory comprising:
a syringe attachment unit configured to be connected to a syringe and to detect pressure data of the syringe; and
a first digital display unit configured to wirelessly connect to the syringe attachment unit, the first digital display unit configured to indicate a pressure corresponding to the pressure data of the syringe.

38. The syringe pressure accessory of claim 37 wherein the syringe attachment unit includes a pressure sensor configured to be connected to the syringe and to detect the pressure data corresponding to pressure in the syringe.

39. The syringe pressure accessory of claim 38 wherein the syringe attachment unit includes a processor connected to the pressure sensor and to analyze the pressure data to determine the pressure in the syringe.

40. The syringe pressure accessory of claim 38 wherein the first digital display unit includes a processor wirelessly connected to the pressure sensor to analyze the pressure data to determine the pressure in the syringe.

41. The syringe pressure accessory of claim 37 wherein the syringe attachment unit includes a non-coiled antenna.

42. The syringe pressure accessory of claim 37 wherein the first digital display unit includes a non-coiled antenna.

43. The syringe pressure accessory of claim 37 further comprising a second digital display unit configured to wirelessly connect to the syringe attachment unit.

44. The syringe pressure accessory of claim 37 wherein the first digital display is configured to indicate the pressure in the syringe in real-time.

45. The syringe pressure accessory of claim 37 wherein the first digital display unit is configured to display a pressure chart based on the pressure data.

46. The syringe pressure accessory of claim 45 wherein the first digital display unit is configured to add a marker to the pressure chart.

47. The syringe pressure accessory of claim 37 wherein the processor is configured to analyze the pressure data and the digital display is configured to display a result of the analysis of the pressure data.

48. A method for determining pressure in multiple syringes comprising:
using a first disposable unit to detect pressure data of a first syringe; and
using a durable unit to analyze the pressure data of the first syringe to determine the pressure in the first syringe.

49. The method of claim 48 further comprising using a disposable membrane and a reusable membrane to detect the pressure data of the first syringe.

50. The method of claim 49 further comprising:
using a second disposable unit to detect pressure data of a second syringe; and
using the durable unit to analyze the pressure data of the second syringe to determine the pressure in the second syringe.

51. The method of claim 50 further comprising disposing of the first disposable unit and the first syringe.

52. A method for determining the pressure of a fluid in a gastric band, the method comprising the steps of:
detecting the pressure of the fluid in the syringe using a syringe pressure accessory having
a disposable membrane in fluid communication with a fluid in a syringe,
a reusable membrane in contact with the disposable membrane for indicating a pressure of the fluid in the syringe,
a reusable pressure sensor connected to the reusable membrane for detecting the indicated pressure of the fluid in the syringe, and
a processor attached to the reusable pressure sensor for analyzing the pressure of the fluid in the syringe;
adjusting the volume of the fluid in the gastric band based on the analysis of the pressure of the fluid in the syringe by injecting fluid from the syringe.

53. A method for treating obesity, the method comprising the steps of:
implanting a gastric band in an obese patient;
determining a first pressure of a fluid in the gastric band using a syringe pressure accessory having
a disposable membrane in fluid communication with a fluid in a syringe,
a reusable membrane in contact with the disposable membrane for indicating a pressure of the fluid in the syringe,
a reusable pressure sensor connected to the reusable membrane for detecting the indicated pressure of the fluid in the syringe, and
a processor attached to the reusable pressure sensor for analyzing the pressure of the fluid in the syringe;
adjusting the volume of the fluid in the gastric band based on the analysis of the pressure of the fluid in the syringe by injecting fluid from the syringe; and
determining a second pressure of the fluid in the gastric band using the syringe pressure accessory, thereby treating obesity.

54. An obesity treatment system for use in conjunction with a gastric band for the treatment of obesity, the system comprising:
an access port connected to the gastric band;
a disposable membrane in fluid communication with a fluid in the access port;
a reusable membrane in contact with the disposable membrane for indicating a pressure of the fluid in the access port;
a reusable pressure sensor connected to the reusable membrane for detecting the indicated pressure of the fluid in the access port; and
a processor attached to the reusable pressure sensor for analyzing the pressure of the fluid in the access port.

55. The system of claim 54 further comprising a volume measuring system connected to the processor, the volume measuring system detecting volume data indicating a volume of fluid added to the access port from a syringe.

56. The system of claim 55 further comprising an electrical parameter measuring system connected to the processor, the electrical parameter measuring system detecting electrical parameter data indicating a location of a syringe relative to the gastric band, a location of the syringe relative to the access port, a status of the access port, a health of a tissue, a tissue layer composition and depth, or a status of fluids in the access port.

57. An obesity treatment system for use in conjunction with a gastric band for the treatment of obesity, the system comprising:
an access port connected to the gastric band;
a catheter connected to the access port;
a syringe connected to the catheter;
a disposable membrane in fluid communication with a fluid in the catheter;
a reusable membrane in contact with the disposable membrane for indicating a pressure of the fluid in the catheter;
a reusable pressure sensor connected to the reusable membrane for detecting the indicated pressure of the fluid in the catheter; and
a processor attached to the reusable pressure sensor for analyzing the pressure of the fluid in the catheter.

58. The system of claim 57 further comprising a volume measuring system connected to the processor, the volume measuring system detecting volume data indicating a volume of fluid added to the access port from the syringe.

59. The system of claim 58 further comprising an electrical parameter measuring system connected to the processor, the electrical parameter measuring system detecting electrical parameter data indicating a location of the syringe relative to the gastric band, a location of the syringe relative to the access port, a status of the access port, a health of a tissue, a tissue layer composition and depth, or a status of fluids in the access port.
